(19) United States
(12) Patent Application Publication
(10) Pub. No.: US 2010/0323976 A1
(43) Pub. Date: Dec. 23, 2010

(54) NOVEL ANTI-INFLAMMATORY PRO-DRUGS

(76) Inventors: Johannes Maria Franciscus Gerardus Aerts, Abcoude (NL); Herman Stevem Overkleeft, Leiden (NL)

Correspondence Address: FOLEY AND LARDNER LLP;SUITE 500, 3000 K STREET NW, WASHINGTON, DC, 20007, US

(21) Appl. No.: 12/664,235

(22) PCT Filed: Jun. 13, 2008
(86) PCT No.: PCT/NL2008/050379

§ 371 Date: Apr. 30, 2010

(30) Foreign Application Priority Data
Dec. 18, 2007 (EP) 07110246.1

(51) Int. Cl. A61K 31/7034; C07J 41/00; A61P 29/00

(52) U.S. Cl. 514/26; 536/5

(57) ABSTRACT

The present invention relates to compounds according to formula (I): wherein R² is absent or a linking moiety and R³ is selected from the group consisting of anti- inflammatory agents and pharmaceutically acceptable salts thereof, pharmaceutical compositions comprising compounds of formula (I) and the use of these pharmaceutical compositions for the treatment or prophylaxis of chronic inflammatory diseases, in particular those that are caused by chronically activated macrophages. The chronic inflammatory disease is in particular atherosclerosis, (rheumatoid) arthritis, an (auto) immune disease or sarcoidosis.

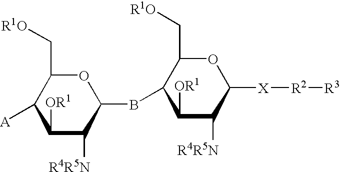

### FIELD OF THE INVENTION

The present invention relates to novel anti-inflammatory pro-drugs comprising a monomeric glucosamine unit or a dimeric moiety of β-1,4-linked glucosamine units which are linked to an anti-inflammatory agent or compound. The novel anti-inflammatory pro-drugs are very suitable for the treatment and prophylaxis of chronic inflammatory diseases.

### BACKGROUND OF THE INVENTION

Chronic inflammatory conditions are generally driven by the presence of chronically activated macrophages at sites of pathological inflammation. These macrophages produce factors that affect other elements of the immune system and promote inflammation and tissue damage. Examples of such diseases include atherosclerosis and (auto)immune diseases, e.g. ulcerative bowel disease, sarcoidosis and arthritis. Treatments according to the prior art imply non-specific and non-targeted suppression of macrophage activation by anti-inflammatory steroid agents, NSAID's (non-steroid anti-inflammatory drugs) and anti-inflammatory proteins.

The major problem associated to these agents is that they have adverse side-affects. In particular, these agents also suppress the immune system at locations in the mammalian body where this is not desired, i.e. at locations where there is no pathological inflammation. Such a non-specific suppression of the immune system leads to adverse effects such as increased vulnerability for infection and reduced hematopoiesis.

It is therefore an object of the present invention to provide anti-inflammatory agents that are more selective and that do not have the systemic adverse effects mentioned above. The inventors discovered that chronically activated macrophages produce a particular glycosidase, i.e. chitotriosidase. Chitotriosidase is disclosed in WO 96/40940, incorporated by reference. Chitotriosidase-producing macrophages occur in for example atherosclerotic lesions, inflammatory joint lesions of patients suffering from (rheumatoid) arthritis and granulomatous tissue in patients suffering from sarcoidosis and inflamed intestine of patients with ulcerative colitis.

### SUMMARY OF THE INVENTION

The present invention relates to novel compounds according to formula (I):

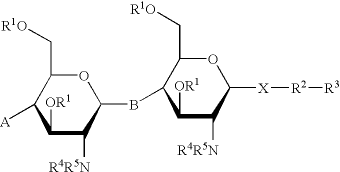

wherein R¹ is selected from the group consisting of hydrogen and OH-protective groups;
R² is absent or is a linking moiety;
R³ is selected from the group consisting of anti-inflammatory agents and pharmaceutically acceptable salts thereof;
X is O or S;
A is selected from the group consisting of hydrogen, —OR¹, —NR⁴R⁵ and

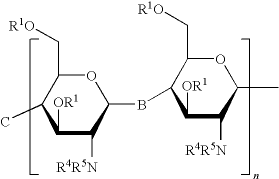

B is selected from the group consisting of —OR¹, —O—, —S—, —NR⁴—, —C(R⁴R⁵)—;
C is selected from the group consisting of hydrogen, —OR¹, —NR⁴R⁵;
R⁴ and R⁵ are independently selected from the group consisting of hydrogen, linear, branched or cyclic C₁-C₆ alkyl groups, linear and branched or cyclic C₂-C₆ alkenyl groups; linear, branched or cyclic C₂-C₁₂ alkynyl groups; C₆-C₁₂ aryl groups, C₇-C₁₂ alkaryl groups and C₇-C₁₂ alkylaryl groups; and
n is in the range of 1-10.

If B is —OR¹, the compounds according to formula (I) obviously contain only one glucosamine unit as will be apparent to the person skilled in the art.

### DETAILED DESCRIPTION OF THE INVENTION

### Definitions

The verb “to comprise” as is used in this description and in the claims and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

A prodrug is to be understood as a compound that is capable of being converted to an active drug. The preparation of prodrugs is for example described in “Design of Prodrugs”, ed. H. Bundgaard, Elsevier, 1985.

Alkyl groups are of the formula CₙH₂ₙ₊₁ and may be linear, branched or cyclic. Suitable examples include methyl, ethyl, 1-butyl, 2-methylpropyl, 1-pentyl, cyclohexyl and the like.

Alkenyl groups are of the formula CₙH₂ₙ₋₁ and may be linear, branched or cyclic.

Suitable examples include ethenyl, 1-butenyl, 2-methylpropenyl, 1-pentenyl, cyclohexenyl and the like. Where appropriate, the alkenyl compounds may have more than one unsaturated carbon-carbon bond, e.g. 1-hex-2-en-4-ynyl and 1-hexa-2,4-diynyl.

Alkynyl groups are of the formula CₙH₂ₙ₋₃ and may be linear, branched and optionally cyclic, although cyclic alkynyl compounds are usually strained and therefore not very stable. Suitable examples include ethynyl, 1-butynyl, 2-methylpropynyl, 1-pentynyl and the like. Where appropriate, the alkynyl groups may have more than one unsaturated carbon-carbon bond, e.g. 1-hex-2-en-4-ynyl and 1-hexa-2,4-diynyl.

The alkyl, alkenyl and alkynyl groups may be substituted with heteroatom containing groups or may be interrupted by one or more heteroatoms. Cyclic alkyl and alkenyl groups may also contain one or more heteroatoms within their ring structure. Suitable examples of such heteroatoms include oxygen, sulphur and nitrogen. Obviously, an alkyl group or an alkenyl group can only be a cyclic group when it contains al least three carbon atoms or two carbon atoms and a heteroatom, e.g. an oxygen atom, so that it represents an oxiranyl group as will be understood by a person skilled in the art. Linear, branched or cyclic alkyl and alkenyl group are therefore hydrocarbyl groups which may optionally be substituted or interrupted with one or more heteroatoms selected from the group consisting of O, S and N. For example, the alkyl group may be methoxy methylene or 2-methoxy butyl as will be apparent to those skilled in the art. If required, such a heteroatom may itself be substituted with a hydrocarbyl group, i.e. an alkyl group, an aryl group, an alkylaryl group or an arylakyl group, so that the alkyl group is for example ethoxy, phenoxy or p-methylphenoxy.

Aryl, alkaryl and alkylaryl groups comprise at least one phenyl or at least one naphtyl group and may be substituted with one or more alkyl, alkenyl or alkynyl groups and/or with one or more heteroatom containing groups. Aryl, alkaryl and alkylaryl groups may also contain one or more heteroatoms within their ring structure. Suitable examples of such heteroatoms include oxygen, sulphur and nitrogen. Suitable examples of aryl groups include phenyl, 4-dimethylaminophenyl, 1-naphtyl and 4-pyridinyl. Suitable examples of alkylaryl groups include benzyl, 4-methylbenzyl and 4-fluorobenzyl. Alkylaryl groups are therefore alkyl groups having one or more aryl groups as substituents. Suitable examples of alkaryl groups include 4-methylphenyl, 4-methoxyphenyl, 4-methoxymethylenephenyl. Alkaryl groups are therefore aryl groups having one or more alkyl groups as substituents.

The alkyl, alkenyl, alkynyl, aryl, alkaryl and alkylaryl may also be substituted with one or more halogen atoms selected from the group consisting of F, Cl, Br and I.

The terms “OH protective group” and “amine protective group” should be understood as a group that is capable to protect an OH-group or an amino group (primary or secondary), respectively, under various reaction conditions including basic, acidic, reducing and oxidising conditions as is well known to the person skilled in the art. Suitable OH protective groups and suitable amine protective groups are well known in the art and are for example disclosed in handbooks such as T. W. Greene, “Protective Groups in Organic Synthesis” (1981), Carey and Sundberg, “Advanced Organic Chemistry, Part B: Reactions and Synthesis” (1977), J. F. W. McOmie, “Protective Groups in Organic Chemistry” (1995) and Peter G. M. Wuts and Theodora W. Greene, “Greene's Protective Groups in Organic Synthesis” (2006). Suitable OH-protective groups include trialkylsilyl ethers, THP-ethers and the like. Suitable amine protective groups include acyl groups, in particular the trifluoroacetyl group, the carbobenzyloxy group, the t-butoxycarbonyl group, the trichloroethoxycarbonyl group and the phthaloyl group.

The term “anti-inflammatory agent” includes not only the active agent per se. The active agent may occur in the form of a salt, a hydrate, a solvate, a polymorph, an enantiomer, a diastereomer, a mesomer, a tautomer, or a combination thereof.

### Release of the Anti-Inflammatory Agent

The compounds according to formula (I) can be hydrolysed by the enzyme chitotriosidase. This enzyme is disclosed in WO 96/40940, incorporated by reference herein. In particular, this enzyme is capable to hydrolyse the monomeric glusoamine unit dimeric moiety of β-1,4-linked glucosamine units of the compounds according to formula (I) thereby releasing the active anti-inflammatory agent. Preferably, the enzyme hydrolyses either the carbon-oxygen bond indicated in the moiety below by the reference number 1 or the R²-oxygen bond indicated by the reference number 2:

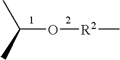

### Preferred Classes of the Compounds According to Formula (I)

According to the invention, preferred compounds according to formula (I) are those wherein the OH-protective groups are independently selected from the group of: linear, branched or cyclic C₁-C₁₂ alkyl groups; linear, branched or cyclic C₂-C₁₂ alkenyl groups; linear, branched or cyclic C₂-C₁₂ alkynyl groups; C₇-C₃₀ arylalkyl groups; silyl groups of the formula —Si(R⁴)₃, wherein each R⁴ is independently selected from the group consisting of a linear, branched or cyclic C₁-C₆ alkyl groups, linear and branched or cyclic C₁-C₆ alkoxy groups; C₆-C₁₂ aryl groups, C₇-C₁₂ alkaryl groups, C₇-C₁₂ alkylaryl groups; R⁵—C(O)O-groups, wherein R⁵ is selected from the group consisting of linear, branched or cyclic C₁-C₆ alkyl groups, linear and branched or cyclic C₂-C₆ alkenyl groups; linear, branched or cyclic C₂-C₁₂ alkynyl groups; C₆-C₁₂ aryl groups, C₇-C₁₂ alkaryl groups, C₇-C₁₂ alkylaryl groups; the alkyl groups, alkenyl groups, alkynyl groups, and alkoxy groups optionally being interrupted with 1-3 hetero-atoms selected from the group consisting of O, N and S or being substituted by hetero-atom containing groups having the formula R⁶—X—, wherein X is O, N or S and R⁶ is selected from the group consisting of hydrogen or linear and branched or cyclic C₂-C₆ alkenyl groups; linear, branched or cyclic C₂-C₁₂ alkynyl groups; C₆-C₁₂ aryl groups, C₇-C₁₂ alkaryl groups, C₇-C₁₂ alkylaryl groups; the aryl groups, alkaryl groups and alkylaryl groups optionally being substituted by hetero-atom containing groups having the formula R⁶—X—, wherein X is O, N or S and R⁶ is selected from the group consisting of hydrogen or linear and branched or cyclic C₂-C₆ alkenyl groups; linear, branched or cyclic C₂-C₁₂ alkynyl groups; C₆-C₁₂ aryl groups, C₇-C₁₂ alkaryl groups, C₇-C₁₂ alkylaryl groups; and the alkyl groups, alkenyl groups, alkynyl groups, alkoxy groups the aryl groups, alkaryl groups and alkylaryl groups optionally being substituted with a halogen, wherein the halogen is independently selected from F, Cl, Br and I.

According to the invention, another group of preferred compounds according to formula (I) are those wherein the amine protective groups are CF₃₋ₚHₚ—C(O)— groups, wherein p is an integer within the range of 0-3. Most preferably, the amine protective group is trifluoroacetyl or acetyl.

According to the invention, it is preferred that in the preferred compounds according to formula (I), the linking moiety R², if present, is a 1,(4+2n) electronic cascade spacer. Such spacers are well known in the art and decompose through a 1,(4+2n)-elimination (n=0, 1, 2, 3, 4, 5 . . . 10; for example 1,6-elimination, 1,8-elimination, or 1,10-elimination). Such linking moieties are known in the art and are for example disclosed in WO 81/01145 and WO 98/13059, all incorporated by reference.

Preferably, the substituent R², if present, is preferably represented by formula (II), wherein:

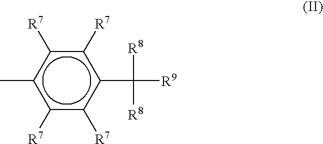

R⁹:

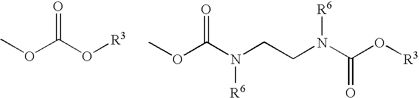

R⁶ is independently selected from the group consisting of hydrogen, C₁-C₆ alkyl, C₆-C₁₂ aryl, C₇-C₁₂ alkaryl groups and C₇-C₁₂ alkylaryl groups;
R⁷ is independently selected from the group consisting of hydrogen, electron-donating groups and electron-withdrawing groups; and
R⁸ is independently selected from the group consisting of hydrogen, C₁-C₆ alkyl, C₆-C₁₂ aryl, C₇-C₁₂ alkaryl groups and C₇-C₁₂ alkylaryl groups.

Electron-donating and electron-withdrawing groups are well known to the person skilled in the art: cf. for example J. March, Advanced Organic Chemistry, 4^{th} Ed., page 280 (Table 9.4) (1992).

### Process for the Preparation of the Compounds According to Formula (I)

The compounds according to formula (I) can be prepared by reacting a precursor of the monomeric glucosamine unit or the dimeric moiety of β-1,4-linked glucosamine units, said precursor comprising a reactive group, with an anti-inflammatory agent comprising a group that is complementary reactive with the reactive group of the precursor of the monomeric glucosamine unit or the dimeric moiety of β-1,4-linked glucosamine units. The stereochemical structure of the compounds according to formula (I) is as follows:

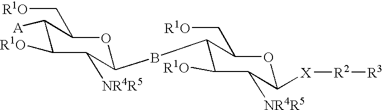

In this document, complementary reactive groups are to be understood as reactive groups that are capable to form, preferably covalent, bonds under conventional reaction conditions as will be apparent to a person skilled in the art. Examples of reactive groups that are complementary reactive are carboxyl and hydroxyl groups that can form an ester group, carboxyl and amine groups that can form an amide group, hydroxy and isocyanate groups that can form a carbamate group, hydroxy groups that can form an ether group etc. However, as will be apparent to those skilled in the art, other modes of molecular bonds, e.g. ionic bonds or coordinative bonds, are in principle within the scope of the present invention, although the formation of a covalent bond is preferred.

Preferably, the precursor of the monomeric glucosamine unit or of the dimeric moiety of β-1,4-linked glucosamine units comprises at least one OH-group that is available for coupling with the anti-inflammatory agent, wherein the anti-inflammatory agent comprises a group that is complementary reactive with an OH-group thereby forming a covalent bond. Suitable examples of combinations of reactive groups and complementary reactive groups are well known to the person skilled in the art and include OH/carboxylic acid groups, OH/carboxylic ester groups, OH/isocyanate groups, OH/OH groups and the like. According to the present invention, it is preferred that the anti-inflammatory agent comprises an OH group.

A representative process for the preparation of the compounds according to the present invention is shown in Scheme 1.

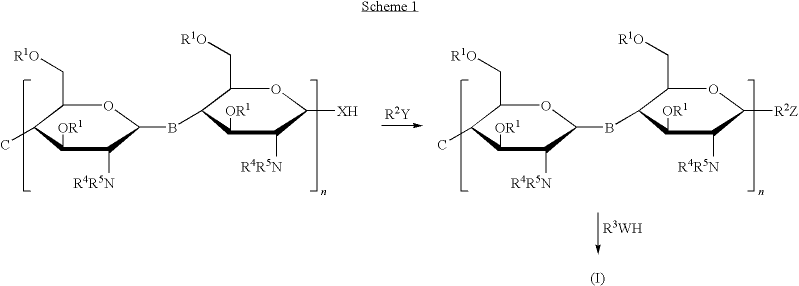

In Scheme 1, the group XH is a reactive group, e.g. an OH group or a carboxylic acid group, which is optionally first derivatised with a linker moiety R²Y, wherein Y is a leaving group. Subsequently, the intermediate is reacted with an anti-inflammatory agent having a reactive group WH that is complementary reactive with Z. Alternatively, the anti-inflammatory agent having a reactive group WH can be reacted with the starting material having the XH group, wherein the group WH is complementary reactive with the group XH. This type of chemistry is well known in the art.

Consequently, the present invention also relates to a process for the preparation of a compound according to formula (I), said process comprising the steps of:
(i) reacting a compound according to formula (III)

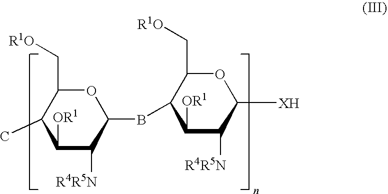

with a reagent R²Y to form an intermediate product according to formula (IV)

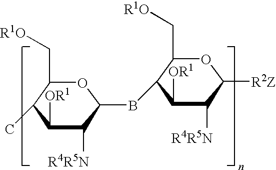

; and
(ii) reacting a compound according to formula (IV) with a reagent R³WH; wherein R¹, R², R³, R⁴ and R⁵ are as defined above.

The process for preparing the compounds according to formula (I) may include one or more protection and deprotection steps if appropriate.

### The Anti-Inflammatory Agent

According to the invention, the anti-inflammatory agent is either a Non-Steroid-Anti-Inflammatory Drug (commonly designated as NSAID) or a steroidal anti-inflammatory agent. Suitable NSAID's include ibuprofen(α-methyl-4-(2-methylpropyl)benzene acetic acid or 2-(4-isobutylphenyl)propionic acid; Merck Index, 13^{th} Ed no. 4906) and diclofenac (2-[(2,6-dichlorophenyl)amino]benzene acetic acid; Merck Index, 13^{th} Ed., no. 3108) which both have a carboxylic group. Suitable steroidal anti-inflammatory agents include prednisone (Merck Index, 13^{th} Ed., no. 7810) and prednisolone (Merck Index, 13^{th} Ed., no. 7807) which both have a OH group. Preferably, the anti-inflammatory agent is a steroidal anti-inflammatory agent ans is most preferably prednisone or prednisolone.

According to the present invention, the compounds according to formula (I) are preferably used for the treatment or prophylaxis of a chronic inflammatory disease, wherein it is preferred that the chronic inflammatory disease is caused by chronically activated macrophages. Most preferably, the macrophages are chitotriosidase producing macrophages. As a consequence, the present invention also relates to a method for the treatment or prophylaxis of a chronic inflammatory disease in a mammal in need thereof, wherein a therapeutically effective amount of a pharmaceutical composition comprising a compound according to formula (I) is administered to the mammal. The pharmaceutical composition preferably comprises a pharmaceutically acceptable carrier.

According to the invention, the chronic inflammatory disease is preferably selected from the group consisting of atherosclerosis, (rheumatoid) arthritis, an (auto)immune disease or sarcoidosis.

A great advantage of the present invention is that the anti-inflammatory agent has a local action instead of a systemic action. The compounds according to the present invention are therefore suitable agents for drug targeting and permit a regulated or controlled drug activation. The anti-inflammatory agent is locally released by chitotriosidase at sites of chronic inflammation. In addition, the release of the anti-inflammatory agent is reduced, interrupted or even discontinued when the inflammation is resolved. Accordingly, the compounds according to the present invention provide a self-controlled method for in particular the treatment of inflammation at sites where the enzyme chitotriosidase is produced.

### Examples

### Example 1

### Direct Coupling to Prednisone Monosaccharide

The synthetic route for preparing a prednisone monosaccharide from glucosamine is shown in Scheme 1.

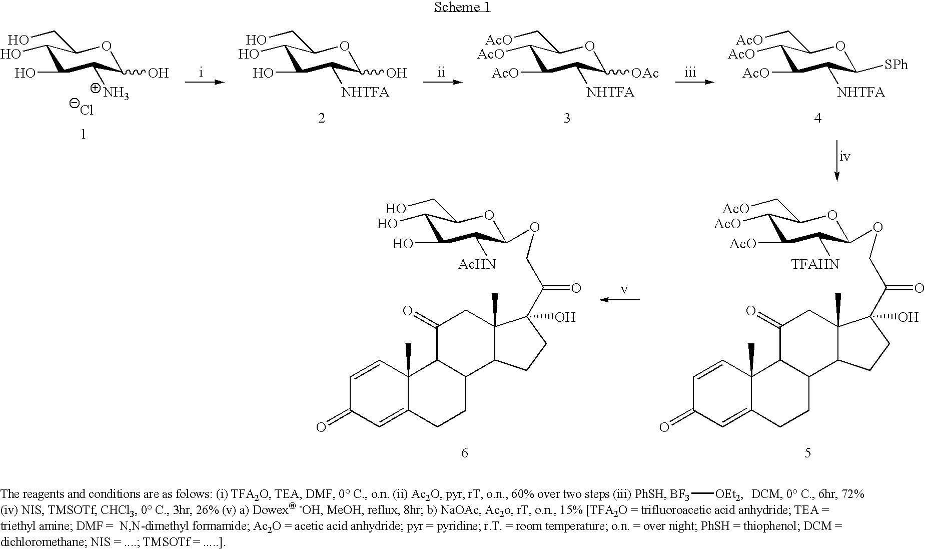

### 2-Trifluoracetamido-2-deoxy-D-glucopyranose (2)

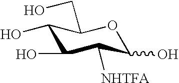

A solution of D-glucosamine hydrochloride (10 g, 46.4 mmol) in DMF (100 mL), was cooled to 0° C. Triethyl amine (14 mL, 140 mmol, 3 equiv.) was added, followed by (7.2 mL, 51 mmol, 1.1 equiv.) trifluoroacetic anhydride in DMF (10 mL). After several minutes, the solution turned slightly orange. The solution was stirred overnight under an Ar-atmosphere. Water (10 mL) was added, and the mixture was taken to dryness by rotary evaporation. The residue was triturated with hot EtOAc and filtered. Silica gel purification (100% EtOAc to 10% MeOH in EtOAc) yielded an off-white solid which was used without further purification. TLC: 20% MeOH in DCM.

### 2-Trifluoracetamido-2-deoxy-1,3,4,6-terta-O-acetyl-D-copyranose (3)

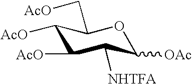

Crude compound 2 was dissolved in Ac₂O/Pyridine (1:3; 100 mL). The mixture was stirred overnight at ambient temperature. The reaction was cooled to 0° C. and quenched with MeOH (25 mL) and diluted with EtOAc (75 mL). Subsequently the reaction mixture was washed with 1M HCl (25 mL), NaHCO₃ (sat. aq.) (25 mL) and brine (25 mL). The organic layer was dried (MgSO₄) and concentrated under reduced pressure. Silica gel purification (30% EtOAc in PE) afforded compound 3 (13.77 g, 31.08 mmol; 60%). TLC: 70% EtOAc in PE. ¹H NMR: (400 MHZ, CDCl₃) δ (ppm) 2.05-2.18 [m, 24H, CH₃, 8× Ac]; 3.91 [dd, J=6.02, 3.77 Hz, 1H, CH, C′-5 β]; 4.07 [d, J=9.70 Hz, 2H, CH₂, C′-6α]; 4.15 [d, J=12.62 Hz, 2H, CH₂, C′-6β]; 4.28 [dd, J=10.66, 2.70 Hz, 1H, CH, C′-5α]; 4.41-4.32 [m, 1H, CH, C′-2β]; 4.52-4.44 [m, 1H, CH, C′-2α]; 5.17-5.10-5.21 [m, 1H, CH, C′-4β]; 5.26-5.19 [m, 1H, CH, C′-4α]; 5.35 [t, J=10.16, 10.16 Hz, 1H, CH, C′-3α/β]; 5.79 [d, J=8.76 Hz, 1H, CH, C′-1β]; 6.26 [d, J=3.46 Hz, 1H, CH, C′-1α]; 6.99 [d, J=13.47 Hz, 1H, NHα]; 7.67 [s, 1H, NHβ]; ¹³C{¹H} NMR(APT) (100 MHz, CDCL₃) δ (ppm) 20.00-20.66 [CH₃, 8× Ac]; 51.62 [CH, C′-2α]; 52.98 [CH, C′-2β]; 61.34 [CH₂, C′-6α]; 61.58 [CH₂, C′-6β]; 67.28 [CH, C′-5α]; 67.91 [CH, C′-5β]; 69.64 [CH, C′-3α]; 70.05 [CH, C′-3β]; 71.89 [CH, C′-4α]; 72.74 [CH, C′-4β]; 89.59 [CH, C′-1α]; 91.57 [CH, C′-1β]; 115.40 [q, CF₃, NTFA α/β]; 157.34 [q, J=16.70 Hz, C_{q}, NTFA α/β]; 171.61-168.29 [C_{q}, 8× Ac].

### Phenyl(2-trifluoracetamido-2-deoxy-3,4,6-tri-O-acetyl-1-thio-D-glucopyranoside) (4)

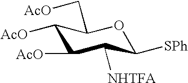

To a solution of 3 (4.43 g, 10 mmol) in DCM (50 mL) thiophenol (1.54 mL, 15 mmol, 1.5 equiv.) and BF₃.Et₂O (3.78 mL, 30 mmol, 3 equiv.) were added. After completion of the reaction, as shown by TLC analysis, NaHCO₃ (sat. aq.) was added. The mixture was extracted with EtOAc (2×150 mL) and washed with H₂O (20 mL) and brine (20 mL). The organic layer was dried over MgSO₄ en concentrated under reduced pressure. Re-crystallisation in EtOAc and PE afforded 4 (3.54 g, 7.17 mmol, 72%) as a yellow solid. TLC: 60% EtOAc in PE. ¹H NMR: (400 MHZ, CDCl₃) δ (ppm) 1.88, 2.00, 2.08 [s, 9H, CH₃, 3× Ac]; 3.79 [d, J=9.67 Hz, 1H, CH, C′-5]; 4.12 [dd, J=19.99, 9.94 Hz, 1H, CH, C′-2]; 4.21 [d, J=1.30 Hz, 2H, C′-6]; 4.78 [d, J=10.41 Hz, 1H, CH, C′-1β]; 5.02 [t, J=9.73, 9.73 Hz, 1H, CH, C′-3,C′-4]; 5.33 [t, J=9.82, 9.82 Hz, 1H CH, C′-3,C′-4]; 7.56-7.21 [m, 5H, CH, arom]; ¹³C{¹H} NMR(APT) (100 MHz, CDCl₃) δ (ppm) 20.72, 20.73, 21.07 [CH₃, 3× Ac]; 53.47 [CH, C′-2]; 62.78 [CH₂, C′-6]; 69.00, 73.92, 76.19 [CH, C′-3, C′-4, C′-5]; 86.49 [CH, C′-1β]; 111.75 [q, J=287.00 Hz, CF₃, NTFA]; 129.15, 129.43 [CH, arom]; 131.77 [C_{q}, arom]; 133.88 [CH, arom]; 157.08 [q, J=38.02 Hz, C_{q}, NTFA]; 169.69, 171.11, 172.05 [C_{q}, 3× Ac]; ESI-MS: 516.1 (M+Na⁺).

### 17α-Hydroxy-3,11,20-trioxo-pregnadien-(1,4)-yl-(21)-2-trifluoracetamido-2-deoxy-3,4,6-tri-O-acetyl-D-gl glucopyranoside (5)

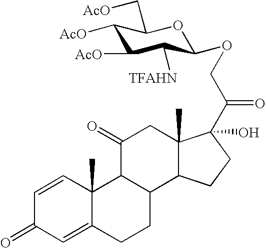

Donor 4 (2.19 g, 4.46 mmol, 2 equiv.; co-evaporated with toluene) and prednisone (0.80 g, 2.23 mmol) were dissolved in anhydrous CHCl₃ (200 mL). The solution was concentrated until precipitation occurred. Then CHCl₃ (20 mL) was added, to redissolve the precipitated prednisone, gaining a concentrated solution of prednisone. After addition of MS 4 Å, the reaction was cooled to 0° C. and stirred for 10 minutes under an Ar-atmosphere. Subsequently, NIS (1.29 g, 5.79 mmol, 2.6 equiv.) and a catalytic amount of TMSOTf were added. After stirring overnight, the reaction was quenched with Na₂S₂O₄ (sat. aq.) and NaHCO₃ (sat. aq.). The mixture was washed with H₂O (75 mL) and brine (75 mL). The CHCl₃ layers were dried (Na₂SO₄), filtered and concentrated. The raw material was purified by Sephadex® size exclusion column chromatography (50 mm D×1500 mm L, eluent MeOH. Evaporation of the eluent afforded title compound 5 (423 mg, 0.571 mmol, 26%) as off-white crystals. TLC: 90% EtOAc in PE. ¹H NMR: (400 MHZ, CDCl₃) δ (ppm) 0.64 [s, 3H, CH₃, C-18]; 1.43 [s, 3H, CH₃, C-19]; 2.01, 2.02, 2.03 [s, 9H, CH₃, 3× Ac]; 2.43-2.35 [m, 2H]; 2.57-2.46 [m, 1H]; 2.76-2.61 [m, 1H]; 2.91 [d, J=12.23 Hz, 1H]; 3.70 [ddd, J=9.96, 4.39, 2.45 Hz, 1H, CH, C′-5]; 4.09 [dd, J=19.12, 8.71 Hz, 1H, CH, C′-2]; 4.20 [dd, J=12.44, 2.33 Hz, 1H, CH₂, C′-6 ]; 4.26 [dd, J=12.45, 4.52 Hz, 1H, CH₂, C′-6]; 4.40 [d, J=18.34 Hz, 1H, CH₂, C-21]; 4.66 [d, J=8.44 Hz, 1H, CH, C′-1β]; 4.83 [d, J=18.36 Hz, 1H, CH₂, C-21]; 5.10 [t, J=9.66, 9.66 Hz, 1H, CH, C′-4]; 5.25 [dd, J=10.34, 9.55 Hz, 1H, CH, C′-3]; 6.08 [s, 1H, C-4]; 6.20 [dd, J=10.24, 1.90 Hz, 1H, C-2]; 7.67 [d, J=10.24 Hz, 1H, C-1]; 7.89 [dd, J=8.46, 3.25 Hz, 1H, NH]; ESI-MS: 742.4 (M+H⁻).

### 17α-Hydroxy-3,11,20-trioxo-pregnadien-(1,4)-yl-(21)-2-oxy-3,4,6-tri-O-acetyl-D-glucopyranoside (6)

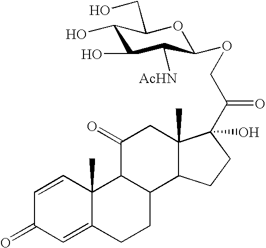

Compound 5 (50 mg, 67 μmol) was dissolved in anhydrous MeOH (6 mL) and freshly prepared Dowex OH (400 mg) was added. The mixture was refluxed for 8 hours, after which it was filtrated and concentrated. The resulting off-white solid was re-dissolved in THF (2 mL) and NaOAc (sat. aq.; 2 mL) and Ac₂O (100 μL, 67 μmol, 1 equiv.) were added. After stirring overnight, at room temperature, an additional equivalent of Ac₂O was added and stirring was continued for 48 hours. The reaction mixture was concentrated and purified by HPLC, yielding target compound 6 (5.5 mg, 9.8 μmol, 15%). ¹H NMR: (600 MHZ, CDCl₃) δ (ppm) 0.64 [s, 3H, CH₃, C-18/C-19]; 1.43 [s, 3H, CH₃, C-19]; 2.01, [s, 3H, CH₃, Ac]; 2.11-2.17 [m, 4H]; 2.43-2.44 [m, 2H]; 2.59-2.63 [m, 1H]; 2.96 [d, J=12.00 Hz, 1H]; 3.19-3.25 [m, 1H, CH, C′-5]; 3.30-3.34 [m, 1H, CH, C′-4]; 3.46 [t, J=10.2 Hz, 1H, CH₂, C′-3]; 3.66 [t, J=10.2 Hz, 1H, CH₂, C′-2]; 3.68 [dd, J=12.0, 2.4 Hz, 1H, CH₂, C′-6]; 3.90 [dd, J=12.0, 2.4 Hz, 1H, CH₂, C′-6]; 4.45 [d, J=9.00 Hz, 1H, CH, C′-1β]; 4.50 [d, J=18.00 Hz, 1H, CH₂, C-21]; 4.70 [d, J=18.00 Hz, 1H, CH₂, C-21]; 6.08 [s, 1H,]; 6.18 [d, J=1.80 Hz, 1H]; 7.67 [d, J=10.24 Hz, 1H]; ESI-MS: 562.1 (M+H⁺).

### Example 2

### Direct Coupling to Prednisone Disaccharide

The synthetic route for preparing a prednisone monosaccharide from glucosamine is shown in Schemes 2 and 3.

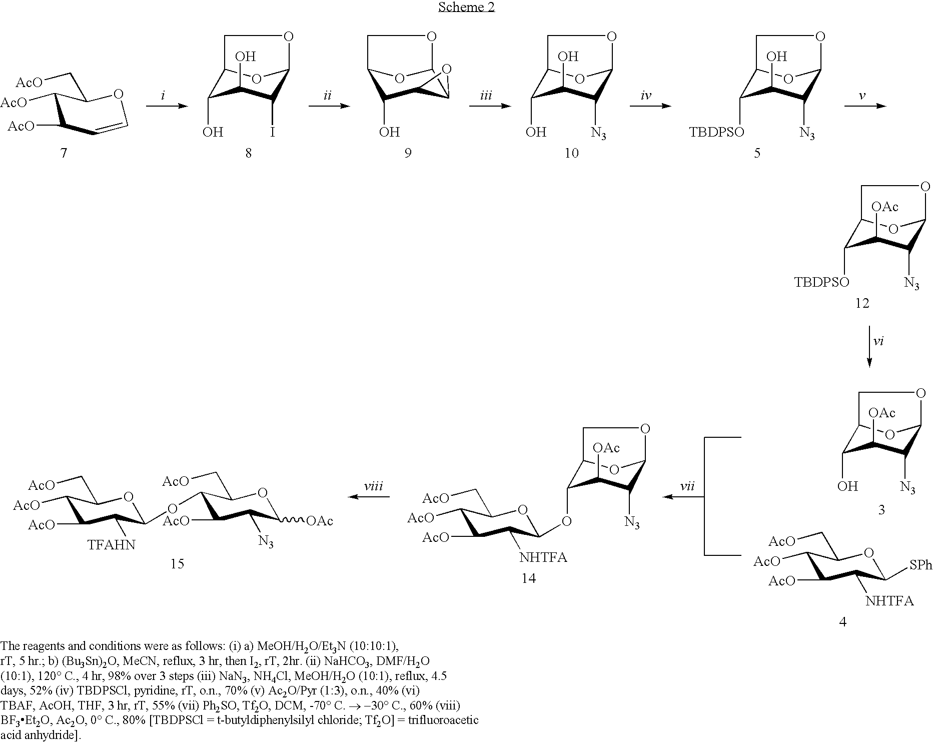

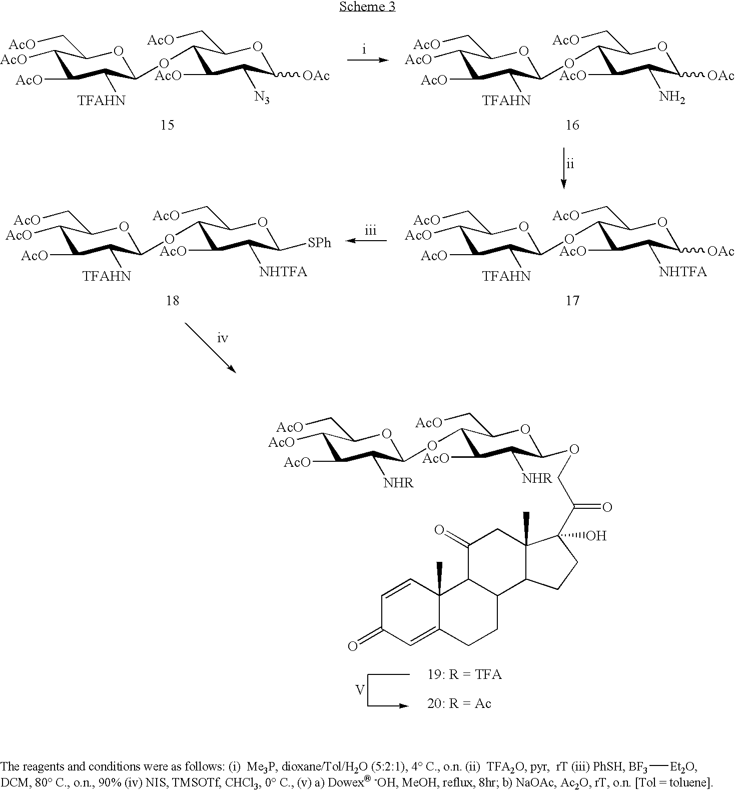

### D-Glucal

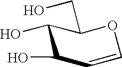

A solution of commercially available tri-O-acetyl-D-glucal (2.72 g, 10 mmol) was dissolved in MeOH—H₂O-Et₃N (10:10:1, 125 mL) and stirred for 1 hour at ambient temperature, followed by removal of all volatiles under reduced pressure. The residue was dried by co-evaporation with dioxane (3×50 mL). The resulting clear oil was used without any further purification.

### 1,6-Anhydro-2-deoxy-β-D-glucopyranose (8)

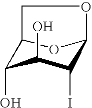

Crude D-Glucal (1.46 g, 10 mmol) was dissolved in MeCN (100 mL). The solution was treated with bis(tributyl stannyl)oxide (4.08 mL, 4.77 g, 8 mmol) and MS4 Å (activated) and refluxed for 2.5 hours. Subsequently, the reaction was cooled to 0° C., followed by portionwise addition of I₂ (3.8 g, 15 mmol, 1.5 equiv.). The dark brown mixture was stirred overnight at +4° C. TLC showed complete conversion of the D-Glucal to 8. The mixture was filtered through Celite and concentrated. To the residue were added Na₂S₂O₃ (50 mL, sat. aq.) and PE (50 mL), and the biphasic mixture was vigorously stirred for several hours until the mixture discoloured. The aqueous phase was washed repeatedly with EtOAc (4×40 mL). The combined organic layers were dried (Na₂SO₄) and concentrated in vacuo. The crude product was used without further purification in the next step.

### 1,6:2,3-Dianhydro-β-D-mannopyranose (9)

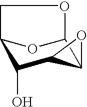

A heterogeneous solution of compound 8 (2.71 g, 10 mmol) and NaHCO₃ (2.5 g, 25 mmol, 2.5 equiv.) in DMF—H₂O (10:1) was heated to 120° C. After 4 h, the reaction mixture was cooled, concentrated (in vacuo) and silica gel purification (0-10% MeOH in EtOAc) yielded title compound 9 (1.42 g, 9.85 mmol, 98% over 3 steps) as a light yellow oil. ¹H NMR: (200 MHZ, CDCl₃) δ (ppm) 3.15 [d, J=3.7 Hz, 1H, CH, C′-2]; 3.25 [d, J=8.8 Hz, 1H, CH, C′-4]; 3.44 [t, J=2.9, 1H, CH, C′-3]; 3.69-3.95 [m, 2H, CH₂, C′-6]; 5.70 [d, J=2.9 Hz, CH, C′-1] ¹³C{¹H} NMR(APT) (50 MHz, CDCl₃) δ (ppm) 48.49 [CH, C′-2]; 53.23 [CH, C′-3]; 64.69 [CH₂, C′-6]; 65.84 [CH, C′-4]; 73.21 [CH, C′-5]; 96.56 [CH, C′-1].

### 1,6-Anhydro-2-azido-2-deoxy-β-D-glucopyranose (10)

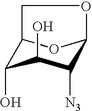

The di-anhydro sugar 9 (1.15 g, 8 mmol) was heated to reflux in a 10:1 MeOH—H₂O (40 mL) solution containing NaN₃ (5.20 g, 80 mmol, 10 equiv.), and NH₄Cl (4.24 g, 80 mmol, 10 equiv.). After ¹H NMR showed complete conversion to the azide 10 (4.5 days), the solution was cooled, filtered through Celite and concentrated under reduced pressure. Silica gel purification (80%-100% EtOAc in PE) yielded title compound 10 as an off-white solid (0.99 g, 5.32 mmol, 66.5%). ¹H NMR: (200 MHZ, CDCl₃) δ (ppm) 3.17 [s, 1H, CH, C′-2]; 3.53 [s, 1H, CH, C′-4]; 3.32-3.69 [m, 2H, CH, CH₂, C′-3, C′-6]; 4.03 [d, J=7.3 Hz, 1H, CH₂, C′-6]; 4.46 [d, J=4.3 Hz, 1H, CH, C′-5]; 5.36 [s, 1H, CH, C′-1] ¹³C{¹H} NMR(APT) (50 MHz, CDCl3) δ (ppm) 64.07 [CH, C′-2]; 66.55 [CH₂, C′-6]; 72.92 [CH, C′-3, C′-4]; 77.99 [CH, C′-5]; 101.79 [CH, C′-1]; ESI-MS: 209.9 (M+Na⁺).

### 1,6-Anhydro-2-azido-4-O-tertbutyldiphenylsilyl-2-deoxy-β-D-glucopyranose (11)

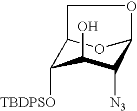

The 1,6-anhydro-2-azido-2-deoxyglucose 10 (0.95 g, 5.08 mmol) was dissolved in pyridine (25 mL) followed by addition of tert-butyldiphenylsilylchloride (1.71 mL, 6.60 mmol). The reaction mixture was stirred overnight, at ambient temperature, after which it was diluted with Et₂O and washed with 1M HCl (25 mL), NaHCO₃ (sat. aq.; 25 mL) and H₂O (25 mL). The organic layer was dried (MgSO₄), filtered and evaporated to dryness. The resulting clear oil was used without any further purification. ESI-MS: 448.1 (M+Na⁺).

### 3-O-Acetyl-1,6-anhydro-2-azido-4-O-tertbutyldiphenylsilyl-2-deoxy-β-D-glucopyranose (12)

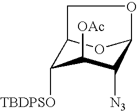

Compound 11 (2.92 g, 5.08 mmol) was dissolved in Ac₂O/Pyridine (1:3; 30 mL), stirred overnight at room temperature and then quenched by addition of MeOH (15 mL) at 0° C. The solution was diluted with EtOAc (30 mL) and washed with 1M HCl (25 mL), NaHCO₃ (sat. aq.; 25 mL) and H₂O (25 mL). The organic layer was dried (MgSO₄) and concentrated in vacuo. Silica gel purification (10% EtOAc in PE) yielded title compound 12 as a colourless oil (0.95 g, 2.03 mmol, 40% over 2 steps). ¹H NMR (200 MHz, CDCl₃) δ (ppm) 1.11 [s, 9H, CH₃, t-Bu]; 1.94 [s, 3H, CH₃, Ac]; 3.11 [s, 1H, CH, C′-2]; 3.49-3.56 [m, 2H, CH, C′-3,C′-6]; 3.68 [dd, J=7.68, 1.03 Hz, 1H, CH, C′-4]; 4.33 [dd, J=6.15, 1.32 Hz, 1H, CH, C′-5]; 4.88 [t, J=1.46 Hz, 1H, CH, C′-6]; 5.46 [s, 1H, CH, C′-1]; 7.34-743 [m, 10H, CH, 2× arom] ¹³C{¹H} NMR(APT) (50 Mhz, CDCl₃) δ (ppm) 18.64 [CH₃, t-Bu]; 26.29 [CH₃, Ac]; 58.59 [CH, C′-2]; 64.35 [CH₂, C′-6]; 69.91, 72.18, 75.71 [CH, C′-3, C′-4, C′-5]; 99.53 [CH, C′-1]; 127.62-135.62 [CH, arom] 132.01, 132.95 [C_{q}, arom]; 168.80 [C_{q}, Ac] IR (neat, cm⁻¹): 702.0, 817.8, 1110.9, 1226.6, 1427.2, 1743.5, 2098.4; ESI-MS: 490.3 (M+Na⁺).

### 3-O-Acetyl-1,6-anhydro-2-azido-2-deoxy-β-D-glucopyranose (13)

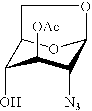

To a stirred solution of 12 (769 mg, 1.70 mmol) in THF (10 mL), AcOH (0.145 mL, 2.55 mmol, 1.5 equiv.) and 1M TBAF in THF (0.76 mL, 3.40 mmol, 2 equiv.) were added. The reaction was stirred overnight followed by dilution with EtOAc (20 mL) and washing with H₂O (10 mL). The organic layer was dried with MgSO₄ and concentrated under reduced pressure. Silica gel purification (30% EtOAc in PE) afforded compound 12 (0.21 g, 0.94 mmol; 55%). ¹H NMR (200 MHz, CDCl₃) δ (ppm) 2.12 [s, 3H, CH₃, Ac]; 3.33 [s, 1H, OH]; 3.45 [s, 1H, CH, C′-6]; 3.64 [s, 1H, CH, C′-4]; 3.82 [dd, J=7.45, 5.93 Hz, 1H, CH, C′-3]; 4.10 [d, J=7.62 Hz, 1H, CH, C′-2]; 4.61 [d, J=5.69 Hz, 1H, CH, C′-5]; 4.85 [d, J=1.49 Hz, 1H, C′-6]; 5.45 [d, J=1.07 Hz, 1H, C′-1] ¹³C{¹H} NMR(APT) (50 MHz, CDCl₃) δ (ppm) 20.83 [CH₃, Ac]; 59.12 [CH, C′-2]; 64.93 [CH₂, C′-6]; 68.47, 72.06, 75.90 [CH, C′-3, C′-4, C′-5]; 99.77 [CH, C′-1]; 169.75 [C_{q}, Ac] IR (neat, cm⁻¹): 613.3, 871.8, 910.3, 1195.8, 1224.7, 1369.4, 1741.6, 2104.2.

### 4-O-(2-trifluoracetamido-2-deoxy-3,4,6-tri-O-acetyl-D-glucopyranosyl)-3-O-acetyl-1,6-anhydro-2-azido-2-deoxy-β-D-glucopyranose (14)

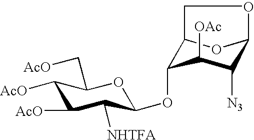

Donor 4 (591 mg, 1.19 mmol, 1.5 equiv. to acceptor; co-evaporated toluene) and Ph₂SO (315 mg, 1.56 mmol, 1.3 equiv. to donor) were dissolved in anhydrous DCM (4 mL). The solution was stirred over 4 Å molecular sieves at ambient temperature for 5 minutes and subsequently cooled to −70° C. followed by activation by addition of Tf₂O (209 μL, 1.26 mmol, 1.05 equiv. to donor). The reaction mixture was allowed to warm to −50° C. and full activation was confirmed by TLC-analysis. Subsequently, the co-evaporated 1,6-anhydro acceptor 13 (183 mg, 0.799 mmol), dissolved in anhydrous DCM (2 mL), was added drop wise to the mixture. During coupling, the temperature of the mixture was raised to −30° C. after which the reaction was quenched by addition of TEA (2 mL). The mixture was allowed to reach room temperature and subsequently diluted with EtOAc (10 mL). The reaction mixture was washed with NaHCO₃ (sat. aq.) (25 mL) and brine (25 mL), after which the organic layer was dried (MgSO₄) and concentrated. The brown oily residue was purified by column chromatography (60% EtOAc in PE) affording compound 14 (303 mg, 0.495 mmol; 60%). ¹H NMR: (500 MHZ, CDCl₃) δ (ppm) 1.99 [s, 3H, CH₃, Ac]; 2.00 [s, 3H, CH₃, Ac]; 2.04 [s, 3H, CH₃, Ac]; 2.07 [s, 3H, CH₃, Ac]; 3.15 [s, 1H, CH, C-2]; 3.65 [s, 1H, CH, C-3]; 3.75 [d, J=7.5 Hz, CH, C-6]; 3.88-3.81 [m, 2H, CH, C′-5, C′-2]; 4.00 [d, J=7.60 Hz, 1H, CH, C-6]; 4.24-4.14 [m, 2H, CH, C′-6]; 4.56 [d, J=5.30 Hz, 1H, CH, C-5]; 5.08 [t, J=9.67, 9.67 Hz, 1H, CH, C′-4]; 5.16 [s, 1H, CH, C-4]; 5.25 [d, J=8.33 Hz, 1H, CH, C′-1β]; 5.42 [s, 1H, CH, C-1]; 5.49 [t, J=9.98, 9.98 Hz, 1H, CH, C′-3]; 7.66 [s, 1H, NH]. ¹³C{¹H} NMR(APT) (125 MHz, CDCl₃) δ (ppm) 20.22 [CH₃,Ac]; 20.37 [CH₃,Ac]; 20.48 [CH₃,Ac]; 20.75 [CH₃,Ac]; [CH, C′-2]; 58.39 [CH, C-2]; 61.81 [CH₂, C′-6]; 64.67 [CH₂, C-6]; 68.38 [CH, C′-4]; 70.01 [CH, C-4]; 71.04 [CH, C′-3]; 71.92 [CH, C′-5]; 73.33 [CH, C-5]; 75.11 [CH, C′-3]; 98.44 [CH, C′-1]; 99.93 [CH, C-1]; 115.41 [q, J=290.21, 289.93, 289.93 Hz, CF₃, TFA]; 157.57 [q, J=37.72, 37.72, 37.44 Hz, C_{q}, TFA]; 169.35 [C_{q}, 2× Ac]; 170.45 [C_{q}, Ac]; 170.71 [C_{q}, Ac]. IR (neat, cm⁻¹): 732.1, 908.9, 1033.1, 1148.9, 1215.2, 1369.3, 1560.2, 1736.0, 2107.5, 3303.6. ESI-MS: 630.2 (M+H₂O).

### 4-O-(2-trifluoracetamido-2-deoxy-3,4,6-tri-O-acetyl-D-glucopyranosyl)-1,3,6-tri-O-acetyl-2-azido-2-deoxy-β-D-glucopyranose (15)

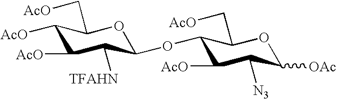

Disaccharide 14 (303 mg, 0.495 mmol) was dissolved in Ac₂O (7.5 mL). The solution was cooled with an ice-bath and stirred for 10 minutes. Subsequently, BF₃.Et₂O (0.313 mL, 2.48 mmol, 5 equiv.) was added drop wise. The reaction was stirred for 1.5 hours after which it was diluted with EtOAc (15 mL) and quenched with NaHCO₃ (sat. aq.; 25 mL). The organic layer was washed with H₂O (40 mL) and brine (20 mL) and subsequently dried over MgSO₄. After concentration (in vacuo), the oily residue was purified on silica gel (40% EtOAc in PE) affording title compound 15 (283 mg, 0.396 mmol; 80%). ¹H NMR: (400 MHZ, CDCl₃) δ (ppm) 2.00-2.25 [m, 36H, CH₃, 2× (6× Ac)]; 3.01 [s, 1H, CH]; 3.08 [s, 1H, CH]; 3.36-4.47 [m, 8H, CH, CH₂]; 4.66 [d, J=8.00 Hz, 1H, CH, C′-1β]; 5.08 [t, J=9.60, 9.60 Hz, 1H, CH]; 5.23 [t, J=9.60, 9.60 Hz, 1H, CH]; 5.40 [t, J=9.60, 9.60 Hz, 1H, CH]; 5.63 [d, J=8.00 Hz, 1H, CH, C-1β]; 6.23 [d, J=8.00 Hz, 1H, CH, C-1α]; 7.61 [d, J=9.20 Hz, 1H, NH]; 7.69 [d, J=8.0 Hz, 1H, NH]. ¹³C{¹H} NMR(APT) (100 MHz, CDCl₃) δ (ppm) 20.12-20.77 [m, CH₃, 6× Ac]; 54.45 [CH, C-2]; 54.57 [CH, C-2]; 61.72 [CH, C′-2]; 61.83 [CH₂, C′-6]; 62.80 [CH₂, C-6]; 67.94-75.09 [m, CH]; 89.74 [CH, C-1α]; 92.15 [CH, C-1β]; 99.84 [CH, C′-1β] 111.21 [q, J=290.21, 289.93, 289.93 Hz, CF₃, TFA]; 156.75 [q, J=37.72, 37.72, 37.44 Hz, C_{q}, TFA]; 168.45-170.91 [C_{q}, 6× Ac. IR (neat, cm⁻¹): 668.0, 930.4, 125.4, 1135.1, 1180.6, 1208.6, 1368.3, 1557.7, 1704.0, 1747.5, 2110.1 ESI-MS: 737.4 (M+Na⁺).

### 4-O-(2-trifluoracetamido-2-deoxy-3,4,6-tri-O-acetyl-D-glucopyranosyl)-2-trifluoro-acetamido-2-deoxy-1,3,6-tri-O-acetyl-D-glucopyranose (17)

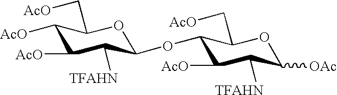

Compound 15 (283 mg, 0.396 mmol) was dissolved in a mixture of dioxane/toluene/H₂O (5:2:1; 8 mL). To the mixture, Me₃P (0.176 mL, 1.98 mmol, 5 equiv.) was added and after stirring for two hours, at ambient temperature, the mixture was concentrated under reduced pressure and co-evaporated several times with toluene, followed by re-dissolving the residue in a mixture of TFAA/pyridine (1:3; 4 mL). This mixture was stirred overnight at ambient temperature after which it was cooled with an ice-bath and quenched by addition of MeOH (10 mL). The resulting solution was diluted with EtOAc (15 mL) and washed with 1 M HCl (25 mL), NaHCO₃ (sat. aq.) (25 mL) and H₂O (25 mL). The organic layer was dried and concentrated in vacuo. Silica gel purification (40% EtOAc in PE) yielded the title compound 17 as an off-white solid (252 mg, 0.321 mmol, 81%). ESI-MS: 807.1 (M+Na⁺).

### Phenyl-4-O-(2-trifluoracetamido-2-deoxy-3,4,6-tri-O-acetyl-D-glucopyranosyl)-2-trifluoracetamido-2-deoxy-3,4,6-tri-O-acetyl-1-thio-D-glucopyranoside (18)

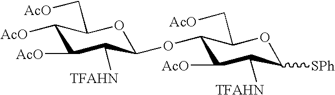

Compound 17 (222 mg, 0.283 mmol) was dissolved in DCM (3 mL). followed by addition of thiophenol (45 μL, 0.424 mmol, 1.5 equiv.) and BF₃.Et₂O (100 μL, 0.848 mmol, 3 equiv.). The reaction was stirred overnight at ambient temperature, after which it was quenched by addition of NaHCO₃ (sat. aq.; 5 mL). The mixture was washed with H₂O (10 mL) and brine (10 mL), followed by drying over MgSO₄ and concentration (in vacuo) of the organic layer. Silica gel purification (60% EtOAc in PE) afforded disaccharide 18 (166 mg, 0.199 mmol; 90%).

### 17α-hydroxy-3,11,20-trioxo-pregnadien-(1,4)-yl-(21)-4-O-(2-trifluoracetamido-2-deoxy-3,4,6-tri-O-acetyl-β-D-glucopyranosyl)-2-trifluoracetamido-2-deoxy-3,6-tri-O-acetyl-β-D-glucopyranose (19)

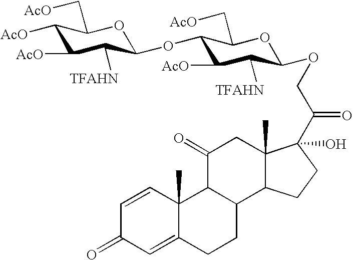

Donor 18 (2.19 g, 4.46 mmol, 2 equiv.; co-evaporated with toluene) and prednisone (0.80 g, 2.23 mmol) were dissolved in anhydrous CHCl₃ (200 mL). The solution was concentrated until precipitation occurred. Then CHCl₃ (20 mL) was added, to redissolve the precipitated prednisone, gaining a concentrated solution of prednisone. After addition of MS4 Å, the reaction was cooled to 0° C. and stirred for 10 minutes under an Ar-atmosphere. Subsequently, NIS (1.29 g, 5.79 mmol, 2.6 equiv.) and a catalytic amount of TMSOTf were added. After stirring overnight, the reaction was quenched with Na₂S₂O₄ (sat. aq.) and NaHCO₃ (sat. aq.). The mixture was washed with H₂O (75 mL) and brine (75 mL). The CHCl₃ layers were dried (Na₂SO₄), filtered and concentrated. The raw m×1500 mm L, eluent MeOH. Evaporation of the eluent afforded title compound 19 (423 mg, 0.571 mmol, 26%) as off-white crystals.

### 17α-hydroxy-3,11,20-trioxo-pregnadien-(1,4)-yl-(21)-4-O-(2-acetamido-2-deoxy-β-D-glucopyranosyl)-2-acetamido-2-deoxy-β-D-glucopyranose (20)

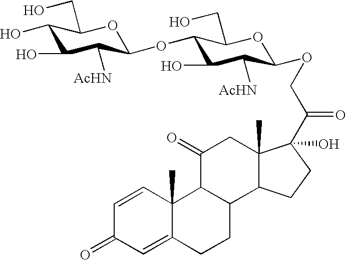

Compound 19 (50 mg, 67 μmol) was dissolved in anhydrous MeOH (6 mL) and freshly prepared Dowex ⁻OH (400 mg) was added. The mixture was refluxed for 8 hours, after which it was filtrated and concentrated. The resulting off-white solid was re-dissolved in THF (2 mL) and NaOAc (sat. aq.; 2 mL) and Ac₂O (100 μL, 67 μmol, 1 equiv.) were added. After stirring overnight, at room temperature, an additional equivalent of Ac₂O was added and stirring was continued for 48 hours. The reaction mixture was concentrated and purified by HPLC, yielding target compound 20 (5.5 mg, 9.8 μmol, 15%).

### Example 3

### Tripartite Prodrug of Prednisone Monosaccharide

The synthetic route for preparing a prednisone monosaccharide from glucosamine is shown in Schemes 4 and 5.

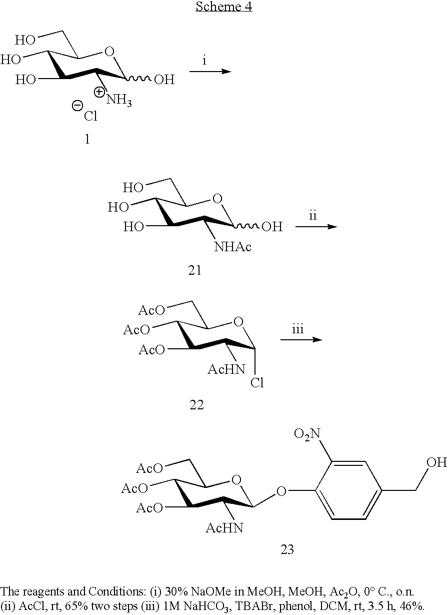

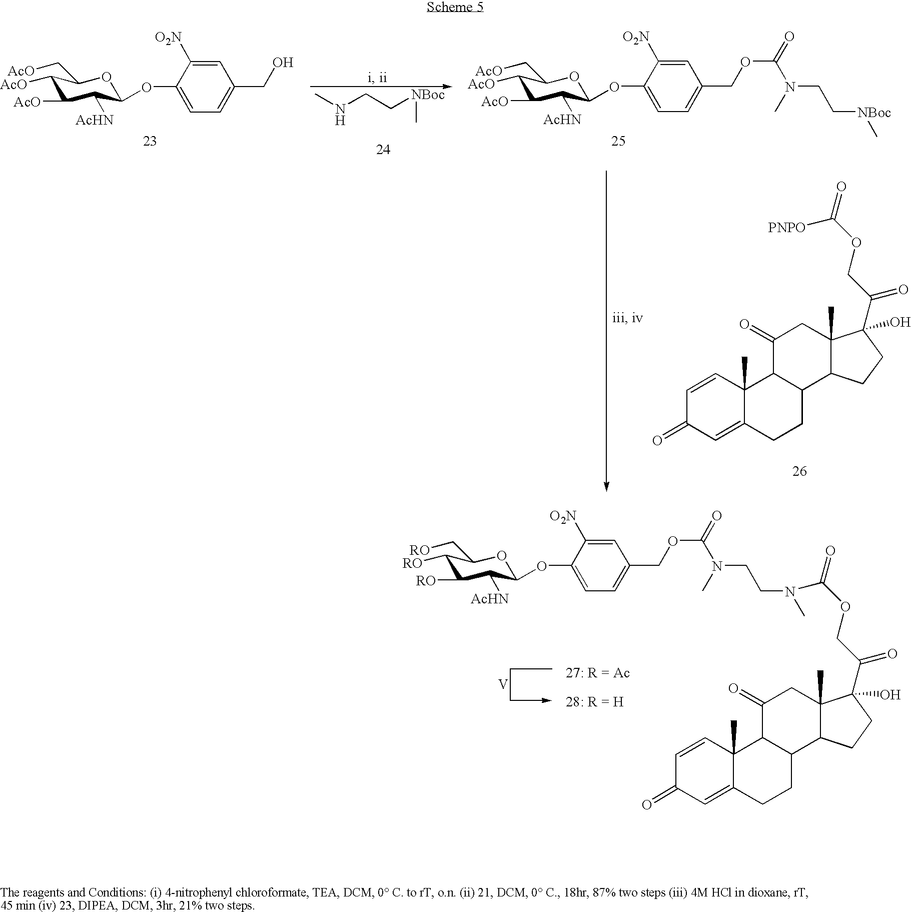

### 2-Acetamido-2-deoxy -D-glucopyranose (21)

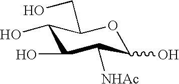

A mixture of MeOH (173 mL) and NaOMe (28 mL, 30% in MeOH) was added to solid D-glucosamine hydrochloride (43.56 g, 200 mmol). The resulting mixture was stirred at ambient temperature for 10 minutes, after which it was gently heated followed by hot filtration. The filtrate was cooled to 0° C. Subsequently, Ac₂O (250 mL) was added and the solution was left overnight at room temperature to crystallize. The crystals were filtered off affording 21 (30 g, 135.7 mmol) as an off-white solid which was used without further purification.

### 2-Acetamido-3,4,6-tri-O-acetyl-2-deoxy-α-D-glucopyranosyl chloride (22)

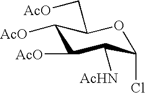

Crude compound 21 (7.5 g, 33.9 mmol) was dissolved in distilled AcCl (25 mL). The reaction mixture started to boil spontaneously after 1 hour. The reaction was left overnight yielding an amber coloured clear liquid. The solution was diluted with DCM (20 mL) and washed within 15 minutes with cold water (40 mL), Na₂CO₃ (sat. aq.) (60 mL) and brine (40 mL). The organic layers were dried (Na₂SO₄) and concentrated under reduced pressure. Crystallization in EtOAc/PE afforded title compound 22 as a fawn solid (8.10 g, 22.20 mmol, 65%). TLC: EtOAc. ¹³C{¹H} NMR(APT) (50 MHz, CDCl₃) δ (ppm) 24.99 [s,CH₃, 2×Ac], 27.22 [s,CH₃, 2×Ac], 57.69 [s,CH, C-2], 65.51 [s,CH₂, C-6], 71.43 [s, CH], 74.34 [s, CH], 75.16 [s, CH], 97.96 [s, CH, C-1], 173.52 [C_{q}, Ac], 174.90 [C_{q}, Ac], 174.93 [C_{q}, Ac], 175.63 [C_{q}, Ac]

### (2-Nitro-4-hydroxymethyl)phenyl-2-acetamido-3,4,6-tri-O-acetyl-2-deoxy-β-D-glucopyranoside (23)

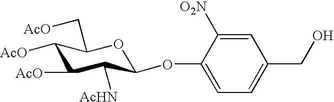

4-Hydroxy-3-nitrobenzyl alcohol (2.53 g, 15 mmol, 1.5 equiv.) and tetra-butylammonium bromide (3.32 g, 10 mmol, 1 equiv.) were dissolved in a two phase-system of DCM (50 mL) and 1M NaHCO₃ (25 mL). The mixture was vigorously stirred for 15 minutes, followed by drop wise addition of compound 22 (3.65 g, 10 mmol), dissolved in DCM (5 mL). Vigorous stirring was continued for 3.5 hours after which the organic layer was washed with H₂O (35 mL) and brine (35 mL). The DCM layer was dried (MgSO₄), filtered and concentrated in vacuo. The resulting yellow oil was purified by silica gel chromatography (EtOAc). Evaporation of the eluent afforded a yellow solid which was re-crystallised (EtOAc/PE) yielding the title compound 23 as yellow crystals (2.29 g, 4.6 mmol, 46%). TLC: 60% EtOAc in PE. ¹H NMR: (600 MHZ, CDCl3) δ (ppm) 1.94 [s, 3H, CH₃, NAc]; 2.01 [s, 3H, CH₃, Ac]; 2.02 [s, 3H, CH₃, Ac]; 2.04 [s, 3H, CH₃, Ac]; 3.89 [d, J=8.4 Hz, 1H, CH, C′-2]; 4.23 [dd, J=12.28, 5.17 Hz, 2H, CH₂, C′-6]; 5.05-5.12 [m, 4H, CH, CH₂, C′-4, C′-5, CH₂]; 5.51 [t, J=8.4 Hz, 1H, CH, C′-3]; 5.58 [d, J=8.04 Hz, 1H, CH, C′-1β]; 7.41-7.75 [m, 3H, CH, arom]; ¹³C{¹H} NMR(APT) (150 MHz, CDCl3) δ (ppm) 20.54, 20.59, 20.64 [CH₃, 3× Ac]; 23.10 [CH₃, NAc]; 55.03 [CH, C′-2]; 61.86 [CH₂, C′-6]; 67.82 [CH₂, CH₂]; 68.55, 71.17, 72.06 [CH, C′-3, C′-4, C′-5]; 99.46 [CH, C′-1]; 120.44, 126.02, 133.54 [CH, arom]; 148.24, 149.52, 154.43 [C_{q}, arom]; 169.23, 170.27, 170.47, 171.20 [C_{q}, 3× Ac, NAc]; IR (neat, cm⁻): 373.8, 463.9, 600.1, 762.1, 791.9, 822.7, 1032.1, 1083.7, 1111.1, 1218.1, 1374.3, 1537.9, 1625.9, 2360.1, 3284.0; ESI-MS: 499.3 (M+H⁺)

### [N,N′-dimethyl]-ethylenediamine-N′-tert-butoxycarbonyl-N-oxycarbonyl-(4-hydroxymethyl-2-nitro)phenyl-2-acetamido-3,4,6-tri-O-acetyl-2-deoxy-β-D-glucopyranoside (25)

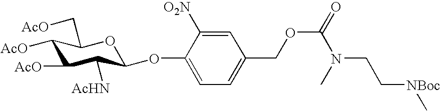

Compound 23 (0.848 g, 1.70 mmol) was dissolved in DCM (15 mL). The solution was cooled using an ice bath followed by drop wise addition of TEA (0.71 mL, 5.1 mmol, 3 equiv.). A solution of 4-nitrophenyl chloroformate (0.50 g, 2.55mmol, 1.5 equiv.), dissolved in DCM (2 mL) was subsequently added over 15 minutes to the reaction. Stirring was continued overnight at ambient temperature. Subsequently, the mixture was cooled using an ice bath and mono-Boc-N,N′-dimethyl-ethylenediamine (0.47 g, 2.55 mmol, 1.5 equiv.) dissolved in DCM (2 mL) was added dropwise. After 18 hours, the reaction was washed with H₂O (10 mL) and brine (15 mL). The organic layer was dried, filtrated and concentrated under reduced pressure. Silica gel purification (1% MeOH in DCM) and evaporation of the eluent afforded title compound 25 as a fawn foam (1.05 g, 1.48 mmol, 87%). ¹H NMR: (400 MHZ, CDCl3) δ (ppm)1.44 [s, 9H, CH₃, t-Bu]; 1.95, 2.05, 2.06, 2.09 [s, 12H, CH₃, Ac]; 2.85 [d, J=22.26 Hz, 3H, CH₃, NMe]; 2.96 [s, 3H, CH₃, NMe]; 3.38 [d, J=15.64 Hz, 4H, CH₂, Et]; 3.93 [td, J=10.30, 8.24, 8.24 Hz, 1H, CH, C′-2]; 4.21 [d, J=12.12 Hz, 1H, CH₂, C′-6]; 4.29 [dd, J=12.28, 5.13 Hz, 1H, CH₂, C′-6]; 5.17-5.07 [m, 1H, CH, CH₂, C′-4, C′-5, CH₂]; 5.57 [d, J=8.55 Hz, 1H, C′-1β]; 5.64-5.58 [m, 1H, CH, C′-3]; 6.31 [s, 1H, NH]; 7.80-7.34 [m, 3H, CH, arom]; ¹³C{¹H} NMR(APT) (150 MHz, CDCl₃) δ (ppm) 21.05-20.22 [CH₃, 3× Ac]; 23.13 [CH₃, NAc]; 28.31 [CH₃, t-Bu]; 34.60 [d, J=30.80 Hz, CH₃, NMe]; 35.28 [CH₃, NMe]; 46.44 [CH₂, Et]; 46.73 [CH₂, Et]; 55.11 [CH, C′-2]; 61.90 [CH₂, C′-6]; 65.25 [dd, J=17.72, 4.17 Hz, CH₂, CH₂]; 68.54 [CH, C′-4, C′-5]; 71.20 [CH, C′-4, C′-5]; 72.11 [CH, C′-3]; 99.34 [CH, C′-1]; 120.47 [CH, arom]; 124.35 [CH, arom]; 132.86 [C_{q}, arom, C_{q}, Boc]; 133.29 [CH, arom]; 141.22 [C_{q}, arom]; 148.96 [C_{q}, arom]; 155.57 [C_{q}, Boc]; 169.38 [C_{q}, NAc]; 170.33 [C_{q}, Ac]; 170.44 [C_{q}, Ac]; 171.13 [C_{q}, Ac]; IR (neat, cm⁻¹): 332.0, 356.3, 374.0, 430.0, 597.7, 1038.0, 1224.2, 1366.0, 1537.8, 1699.8, 1747.1; ESI-MS: 713.4 (M+H⁺)

### Prednisone 21-(p-nitrophenyl carbonate) (26)

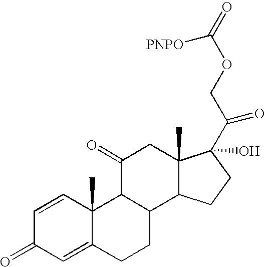

Prednisone (1.79 g, 5 mmol) was dissolved in anhydrous CHCl₃ (25 mL). The solution was cooled using an ice bath, after which a solution of 4-nitrophenyl chloroformate (1.51 g, 6 mmol, 1.2 equiv.) in CHCl₃ (4 mL) was added, over 15 minutes. The milky solution was stirred for 1 hour, followed by addition of pyridine (1.21 mL, 15 mmol, 3 equiv.). When the reaction turned clear the mixture was co-evaporated 3 times with toluene (20 mL), yielding an off-white solid which was used without any further purification.

### 17α-hydroxy-3,11,20-trioxo-pregnadien-(1,4)-yl-(21)-oxycarbonyl-N-[N,N′-dimethyl]-ethylenediamine-N′-tert-butoxycarbonyl-N-oxycarbonyl-(4-hydroxymethyl-2-nitro)phenyl-[4-O-(2-acetamido-3,4,6-tri-O-acetyl-2-deoxy-β-D-glucopyranosyl)-2-acetamido-3,4,6-tri-O-acetyl-2-deoxy-β-D-glucopyranosyl] (27)

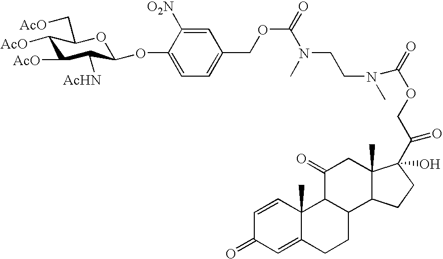

Compound 25 (0.981 g, 1.38 mmol) was dissolved in a cooled (0° C.) solution of 4M HCl in dioxane (7 mL). After 45 minutes, TLC-analysis showed consumption of the starting material. The reaction-mixture was co-evaporated with toluene (10 mL) yielding a white foam. This white foam was dissolved in anhydrous DCM (5 mL) and cooled in an ice bath. A slurry of prednisone derivative 26 in anhydrous DCM (7 mL) was added drop wise followed by addition of DIPEA (0.2 mL, 1.38 mmol, 1 equiv.). The milky reaction mixture was stirred for 3 hours, while it reached room temperature. The resulting clear ochre coloured solution was concentrated in vacuo and applied to a Sephadex® size exclusion column chromatography (50 mmD×1500 mmL) and eluted with MeOH yielding an off-white solid. Further purification was done by column chromatography (2.5% EtOH in CHCl3) and evaporation of the eluent afforded the title compound 27 as an off-white solid (292 mg, 0.29 mmol, 21%). ¹H NMR: (400 MHZ, CDCl₃) δ (ppm) 0.66 [s, 3H, CH₃, C′-18]; 1.43 [s, 3H, CH₃, C′-19]; 1.93, 2.04, 2.08 [s, 12H, CH₃, 4× Ac]; 2.25-2.70 [m, 5H]; 2.86-2.95 [m, 6H, CH₃, 2× Me]; 3.44 [s, 4H, 2× CH₂]; 3.94 [s, 1H, CH, C-2]; 4.18-4.29 [m, 2H, CH₂, C-6]; 4.57 [m, 3H]; 4.91-4.96 [m, 1H]; 5.04-5.17 [m, 2H, CH, C-5, C-4, CH]; 5.57-5.59 [m, 2H, CH, C-3, C-1]; 6.06 [s, 1H]; 6.18 [d, J=10.4 Hz, 1H]; 6.80 [s, 1H, NH]; 7.32-7.83 [m, 4H] IR (neat, cm⁻¹): 312.1, 326.0, 340.0, 376.1, 435.8, 507.9, 602.0, 668.1, 765.6, 822.9, 889.9, 1040.2, 1218.0, 1366.7, 1537.8, 1660.9, 1699.8, 2360.2, 2945.7

### 17α-hydroxy-3,11,20-trioxo-pregnadien-(1,4)-yl-(21)-oxycarbonyl-N-[N,N′-dimethyl]-ethylenediamine-N-oxycarbonyl-4-hydroxy-3-nitro-benzyl-O-[4-O-(2-acetamido-2-deoxy-β-D-glucopyranosyl)-2-acetamido-2-deoxy-β-D-glucopyranose] (28)

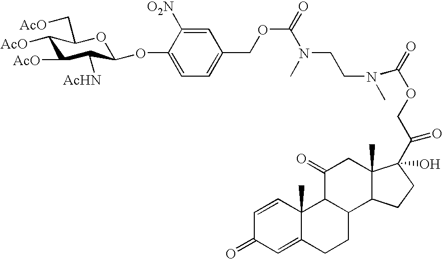

Compound 27 (50 mg, 67 μmol) was dissolved in anhydrous MeOH(6 mL) and freshly prepared Dowex ⁻OH (400 mg) was added. The mixture was refluxed for 8 hours, after which it was filtrated and concentrated. The resulting off-white solid was re-dissolved in THF (2 mL) and NaOAc (sat. aq.; 2 mL) and Ac₂O (100 μL, 67 μmol, 1 equiv.) were added. After stirring overnight, at room temperature, an additional equivalent of Ac₂O was added and stirring was continued for 48 hours. The reaction mixture was concentrated and purified by HPLC, yielding target compound 28 (5.5 mg, 9.8 μmol, 15%).

### Example 4

### Tripartite Prodrug of Prednisone Disaccharide

The synthetic route for preparing a prednisone monosaccharide from glucosamine is shown in Scheme 6.

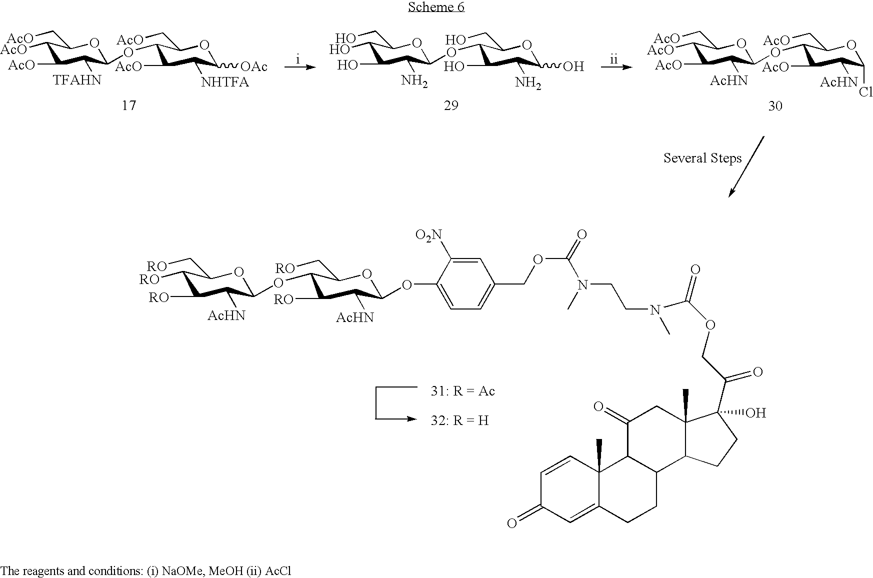

### Example 5

### Method to Detect Conversion of Pro-Drug into Active Component

Any synthesized pro-drug is incubated with 1 mg recombinant human chitotriosidase (produced as described in: van Eijk M, van Roomen C P, Renkema G H, Bussink A P, Andrews L, Blommaart E F, Sugar A, Verhoeven A J, Boot R G, Aerts J M,. “Characterization of human phagocyte-derived chitotriosidase, a component of innate immunity”, Int Immunol. 2005 November; 17(11):1505-12) in 0.1 M potassium phosphate buffer (pH 6.5) for 1 hour at 37° C. The reaction is stopped on ice and formed products are analysed by appropriate methods. In most cases state-of-the art analysis of compounds by LC-MS/MS will be the method of choice. Released chitobiose can be detected by HPLC as described in Aguilera B, Ghauharali-van der Vlugt K, Helmond M T, Out J M, Donker-Koopman W E, Groener J E, Boot R G, Renkema G H, van der Marel G A, van Boom J H, Overkleeft H S, Aerts J M, “Transglycosidase activity of chitotriosidase: improved enzymatic assay for thehuman macrophage chitinase”, J. Biol. Chem. 2003 Oct. 17; 278(42):40911-6.

1. A compound according to formula (I): 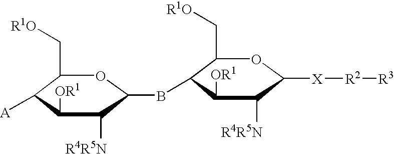
wherein R¹ is selected from the group consisting of hydrogen and OH-protective groups; the OH-protective groups being independently selected from the group consisting of:
linear, branched or cyclic C₁-C₁₂ alkyl groups;
linear, branched or cyclic C₂-C₁₂ alkenyl groups;
linear, branched or cyclic C₂-C₁₂ alkynyl groups;
C₇-C₃₀ arylalkyl groups;
silyl groups of the formula —Si(R⁴)₃, wherein each R⁴ is independently selected from the group consisting of a linear, branched or cyclic C₁-C₆ alkyl groups, linear and branched or cyclic C₁-C₆ alkoxy groups; C₆-C₁₂ aryl groups, C₇-C₁₂ alkaryl groups, C₇-C₁₂ alkylaryl groups;
R⁵—C(O)O-groups, wherein R⁵ is selected from the group consisting of linear, branched or cyclic C₁-C₆ alkyl groups, linear and branched or cyclic C₂-C₆ alkenyl groups;
linear, branched or cyclic C₂-C₁₂ alkynyl groups; C₆-C₁₂ aryl groups, C₇-C₁₂ alkaryl groups, C₇-C₁₂ alkylaryl groups;
the alkyl groups, alkenyl groups, alkynyl groups, and alkoxy groups optionally being interrupted with 1-3 hetero-atoms selected from the group consisting of O, N and S or being substituted by hetero-atom containing groups having the formula R⁶—X—, wherein X is O, N or S and R⁶ is selected from the group consisting of hydrogen or linear and branched or cyclic C₂-C₆ alkenyl groups; linear, branched or cyclic C₂-C₁₂ alkynyl groups; C₆-C₁₂ aryl groups, C₇-C₁₂ alkaryl groups, C₇-C₁₂ alkylaryl groups;
the aryl groups, alkaryl groups and alkylaryl groups optionally being substituted by hetero-atom containing groups having the formula R⁶—X—, wherein X is O, N or S and R⁶ is selected from the group consisting of hydrogen or linear and branched or cyclic C₂-C₆ alkenyl groups; linear, branched or cyclic C₂-C₁₂ alkynyl groups; C₆-C₁₂ aryl groups, C₇-C₁₂ alkaryl groups, C₇-C₁₂ alkylaryl groups; and
the alkyl groups, alkenyl groups, alkynyl groups, alkoxy groups the aryl groups, alkaryl groups and alkylaryl groups optionally being substituted with a halogen, wherein the halogen is independently selected from F, Cl, Br and I;
R² is absent or is a linking moiety, wherein the substituent R², if present, is represented by formula (II), wherein: 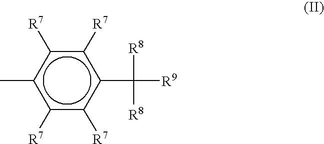
R⁹: 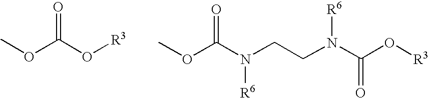
R⁶ is independently selected from the group consisting of hydrogen, C₁-C₆ alkyl, C₆-C₁₂ aryl, C₇-C₁₂ alkaryl groups and C₇-C₁₂ alkylaryl groups;
R⁷ is independently selected from the group consisting of hydrogen, electron-donating groups and electron-withdrawing groups; and
R⁸ is independently selected from the group consisting of hydrogen, C₁-C₆ alkyl, C₆-C₁₂ aryl, C₇-C₁₂ alkaryl groups and C₇-C₁₂ alkylaryl groups;
R³ is selected from the group consisting of anti-inflammatory agents and pharmaceutically acceptable salts thereof;
X is O or S;
A is selected from the group consisting of hydrogen, —OR', —NR⁴R⁵ and 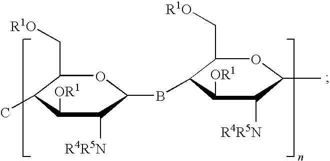
B is selected from the group consisting of —OR¹ (when the compounds according to formula (I) contain only one glucosamine unit), —O—, —S—, —NR⁴—, —C(R⁴R⁵)—;
C is selected from the group consisting of hydrogen, —OR¹, —NR⁴R⁵;
R⁴ and R⁵ are independently selected from the group consisting of amine protective groups, hydrogen, linear, branched or cyclic C₁-C₆ alkyl groups, linear and branched or cyclic C₂-C₆ alkenyl groups; linear, branched or cyclic C₂-C₁₂ alkynyl groups; C₆-C₁₂ aryl groups, C₇-C₁₂ alkaryl groups and C₇-C₁₂ alkylaryl groups; the amine protective groups being selected from R¹⁰—O—C(O)— groups, wherein R¹⁰ is selected from the group consisting of linear, branched or cyclic C₁-C₆ alkyl groups, linear and branched or cyclic C₂-C₆ alkenyl groups; linear, branched or cyclic C₂-C₁₂ alkynyl groups; C₆-C₁₂ aryl groups, C₇-C₁₂ alkaryl groups, C₇-C₁₂ alkylaryl groups, the alkyl groups, alkenyl groups, alkynyl groups, aryl groups, alkaryl groups and alkylaryl groups optionally being substituted with a halogen, wherein the halogen is independently selected from F, Cl, Br and I; and
n is in the range of 1-10.

2. The compound according to claim 1, wherein the anti-inflammatory agent comprises a group that is complementary reactive with an OH-group.

3. The compound according to claim 1, wherein the anti-inflammatory agent comprises a HO-group.

4. The compound according to claim 1, wherein the anti-inflammatory agent is a NSAID.

5. The compound according to claim 1, wherein the anti-inflammatory drug is a steroidal anti-inflammatory agent.

6. A pharmaceutical composition comprising a compound according to claim 1 and a pharmaceutically acceptable carrier.

7. A method for the treatment or prophylaxis of a chronic inflammatory disease comprising administering to a patient in need thereof a compound according to claim 1.

8. (canceled)

9. The method according to claim 7, wherein the chronic inflammatory disease is caused by chronically activated macrophages.

10. The method according to claim 9, wherein the macrophages are chitotriosidase producing macrophages.

11. The method according to claim 7, wherein the chronic inflammatory disease is atherosclerosis, arthritis, an immune disease or sarcoidosis.

12. A process for the preparation of a compound according to formula (I), said process comprising the steps of:
(i) reacting a compound according to formula (III)

13. A method of treating a subject suffering from inflammation comprising administering to a subject in need of such treatment a prodrug comprising an inflammatory agent bound to a moiety, which moiety is recognized and cleaved by a glycosidase produced by activated macrophages present in a site of chronic inflammation, thus releasing the inflammatory agent at the site of chronic inflammation.

14. The method of claim 13 in which the moiety comprises a glycoside.

15. The method of claim 14 in which the glycoside has a structure falling within a formula I.

16. The method of claim 13 in which the site of chronic inflammation is intestine, a joint, or both.

17. The method of claim 13 in which the glycosidase is chitotriosidase.
